(19) United States
(12) Patent Application Publication
(10) Pub. No.: US 2016/0310144 A1
(43) Pub. Date: Oct. 27, 2016

(54) ATRIAL-APPENDAGE LIGATION SURGICAL TOOL

(71) Applicant: OLYMPUS CORPORATION, Tokyo (JP)

(72) Inventors: Takehiro KIMURA, Tokyo (JP); Kazutoshi KUMAGAI, Tokyo (JP); Yoshiro OKAZAKI, Tokyo (JP); Naoya SUGIMOTO, Tokyo (JP); Hiroki HIBINO, Tokyo (JP); Yoshiyuki KUMADA, Tokyo (JP); Hiroshi FUKUDA, Tokyo (JP)

(73) Assignee: OLYMPUS CORPORATION, Tokyo (JP)

(21) Appl. No.: 15/203,532

(22) Filed: Jul. 6, 2016

Related U.S. Application Data
(63) Continuation of application No. PCT/JP2015/054029, filed on Feb. 13, 2015.

(30) Foreign Application Priority Data
Feb. 28, 2014 (JP) 2014-039816

(51) Int. Cl. A61B 17/12

(52) U.S. Cl. CPC A61B 17/12013; A61B 2017/00243

(57) ABSTRACT

An atrial appendage ligation surgical tool that includes: a tubular loop shaft having an inner hole that penetrates therethrough in a longitudinal direction; and a ligature loop that is in an inserted state in the inner hole of the loop shaft and formed of a wire that forms a loop closed with a knot at a distal end of the loop shaft, the loop being contractible in size by being pulled from a proximal end of the loop shaft. The ligature loop is given a tendency to form a flat ring-shaped loop having a size an atrial appendage is insertable when in a free state, and the knot is placed near an end of the loop in a long axis direction thereof.

### CROSS-REFERENCE TO RELATED APPLICATIONS

This is a continuation of International Application PCT/JP2015/054029, with an international filing date of Feb. 13, 2015, which is hereby incorporated by reference herein in its entirety. This application claims the benefit of Japanese Patent Application No. 2014-039816, filed on Feb. 28, 2014, the content of which is incorporated herein by reference.

### TECHNICAL FIELD

The present invention relates to an atrial-appendage ligation surgical tool.

### BACKGROUND ART

Conventionally, one known technique for ligating an atrial appendage involves use of a surgical tool for ligating an atrial appendage. With this surgical tool, after a ligature loop supported at a distal end of a shaft is put around an atrial appendage while the loop is spread wide open held by other supporting members at a plurality of points spaced in the circumferential direction of the ligature loop, the ligature loop is tightened to ligate the atrial appendage (for example, refer to PTL 1).

Since these other supporting members hold the ligature loop by merely hooking the ligature loop at distal end portions formed by spirally wound wires, the supporting members can be easily detached from the ligature loop after ligation.

### CITATION LIST

### Patent Literature

### {PTL 1}

United States Patent Application Publication No. 2008/0294175 specification.

### SUMMARY OF INVENTION

### Technical Problem

The knot in the ligature loop ligating the atrial appendage is relatively large and the cut surface of the ligature loop may be sharp depending on how the cut was made after ligation. There is a possibility that the surrounding tissue is irritated.

The present invention is an atrial-appendage ligation surgical tool, with which a knot in a ligature loop ligating the vicinity of the base of an atrial appendage can be placed at a position where it rarely irritates the surrounding tissue.

### Solution to Problem

An aspect of the present invention provides an atrial-appendage ligation surgical tool, the surgical tool including a tubular loop shaft having an inner hole that penetrates therethrough in a longitudinal direction; and a ligature loop arranged to be in an inserted state in the inner hole of the loop shaft and formed of a wire that forms a loop closed with a knot at a distal end of the loop shaft, the loop being contractible in size by being pulled from a proximal end of the loop shaft, wherein the ligature loop is given a tendency to form a flat ring-shaped loop having a size an atrial appendage is insertable when in a free state, and the knot is placed near an end of the loop in a long axis direction thereof.

According to this aspect, after the loop of the ligature loop at the distal end of the loop shaft guided into the pericardium is put around the atrial appendage, the loop is contracted by pulling the ligature loop from the proximal end of the loop shaft so as to ligate the atrial appendage. In this case, when the atrial appendage is being inserted into the loop, which is formed to have a flat ring shape in a free state, insertion is carried out while the flat loop maintains its topological relationship with respect to the flat atrial appendage.

Another aspect of the present invention provides an atrial-appendage ligation surgical tool, the surgical tool including a tubular loop shaft having an inner hole that penetrates therethrough in a longitudinal direction; and a ligature loop arranged to be in an inserted state in the inner hole of the loop shaft and formed of a wire that forms a loop closed with a knot at a distal end of the loop shaft, the loop being contractible in size by being pulled from a proximal end of the loop shaft, wherein the ligature loop is given a tendency to form a flat ring-shaped loop having a size an atrial appendage is insertable when in a free state, and the knot is placed near an end of the loop in a short axis direction.

According to this aspect, after the loop of the ligature loop at the distal end of the loop shaft guided into the pericardium is put around the atrial appendage, the loop can be contracted by pulling the ligature loop from the proximal end of the loop shaft so as to ligate the atrial appendage. In this case, when the atrial appendage is inserted into the loop formed to have a flat ring shape in a free state and the distal end of the loop shaft is advanced from the tip side of the atrial appendage toward the base side through a gap between the atrial appendage and the pericardial sac along the area near the center of the atrial appendage in the width direction, the flat atrial appendage is inserted into the flat loop while maintaining the topological relationship as the loop is dragged.

Yet another aspect of the present invention provides an atrial-appendage ligation surgical tool, the surgical tool including a tubular loop shaft having an inner hole that penetrates therethrough in a longitudinal direction; a ligature loop arranged to be in an inserted state in the inner hole of the loop shaft and formed of a wire that forms a loop closed with a knot at a distal end of the loop shaft, the loop being contractible in size by being pulled from a proximal end of the loop shaft; and a bifurcated pressing part disposed at the distal end of the loop shaft, and having two rod-shaped contact portions that can be placed at positions that allow pinching of an atrial appendage in a thickness direction, the contact portions branching from the loop shaft side toward the distal end, wherein the knot in the ligature loop is placed near a branching point of the contact portions.

According to this aspect, after the bifurcated pressing part at the distal end of the loop shaft guided into the pericardium is placed so that the atrial appendage is pinched between the two rod-shaped contact portions, and the loop of the ligature loop at the distal end of the loop shaft is put around the atrial appendage, the ligature loop is pulled from the proximal end of the loop shaft so that the loop is contracted and the atrial appendage is ligated.

### BRIEF DESCRIPTION OF DRAWINGS

FIG. 1 is a perspective view of an atrial-appendage ligation surgical tool according to an embodiment of the present invention.

FIG. 2 is a front view showing the relationship between a loop and a knot of the atrial-appendage ligation surgical tool shown in FIG. 1 and an atrial appendage.

FIG. 3 is a front view showing a state in which the loop of the atrial-appendage ligation surgical tool shown in FIG. 1 is brought near the atrial appendage.

FIG. 4 is a front view showing a state in which the loop is put around the atrial appendage from the state shown in FIG. 3.

FIG. 5 is a front view showing a state in which the atrial appendage is ligated by contracting the loop from the state shown in FIG. 4.

FIG. 6 is a side view showing the atrial-appendage ligation surgical tool shown in FIG. 1 when the surgical tool is in a state shown in FIG. 5.

FIG. 7 is a diagram showing a first modification of the atrial-appendage ligation surgical tool shown in FIG. 1.

FIG. 8 is a front view showing a state in which a loop of the atrial-appendage ligation surgical tool shown in FIG. 7 is put around an atrial appendage.

FIG. 9 is a front view showing a state in which a knot in the loop of the atrial-appendage ligation surgical tool shown in FIG. 7 is placed at a final ligation position.

FIG. 10 is a diagram showing a second modification of the atrial-appendage ligation surgical tool shown in FIG. 1.

FIG. 11 is a front view showing a state in which the loop of the atrial-appendage ligation surgical tool shown in FIG. 10 is put around the atrial appendage.

### DESCRIPTION OF EMBODIMENT

An atrial-appendage ligation surgical tool 1 according to one embodiment of the present invention is described below with reference to the drawings.

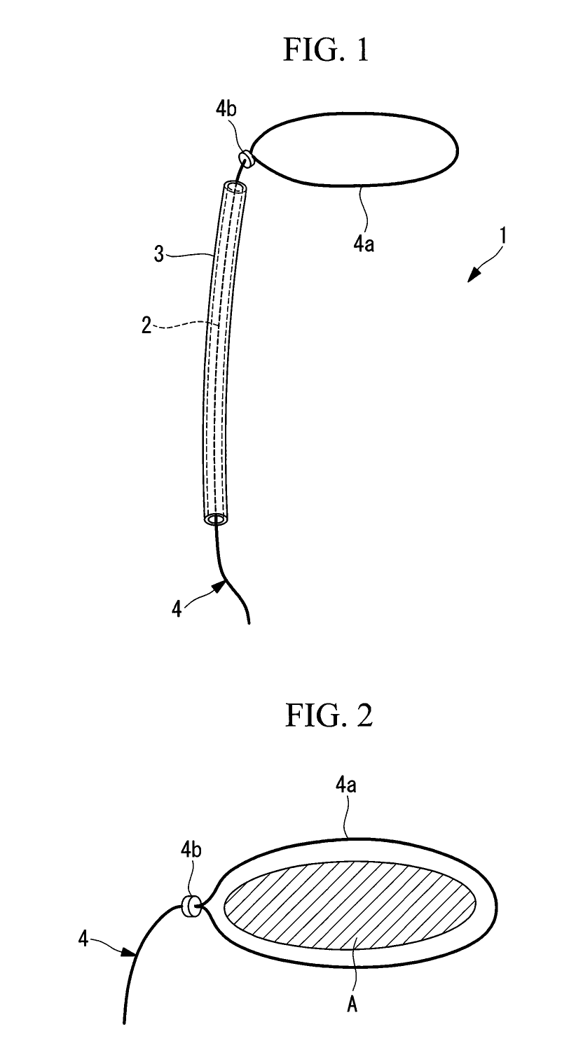

As shown in FIG. 1, the atrial-appendage ligation surgical tool 1 according to this embodiment includes a long tubular loop shaft 3 having an inner hole 2 penetrating therethrough in the longitudinal direction, and a ligature loop 4 accommodated to be in an inserted state in the inner hole 2 of the loop shaft 3.

The loop shaft 3 can bend along the shape of a sheath (not shown) that penetrates through the body surface tissue so that the distal end thereof is placed in a pericardium, and has a rigidity that enables transmission of a pressing force acting in the longitudinal direction applied from the proximal end of the sheath.

The ligature loop 4 is formed of a wire, penetrates through the inner hole 2 of the loop shaft 3, and extends outward from two ends of the loop shaft 3. The section of the ligature loop 4 that extends from the distal end of the loop shaft 3 is turned once to form a single loop 4a, and the distal end thereof is formed into a knot 4b located at an intermediate position in the length direction of the ligature loop itself.

The knot 4b is configured such that the ligature loop 4 does not easily move in a direction causing widening of the loop 4a but can move in a direction causing narrowing of the loop 4a when the other end of the ligature loop 4 extending from the proximal end of the loop shaft 3 is pulled.

In this embodiment, as shown in FIG. 1, the loop 4a of the ligature loop 4 is given a tendency to form a flat ring shape in a free state in which it is free of any external force acting, other than gravity. As shown in FIG. 2, the short axis of the ring shape of the loop 4a is set to be larger than the thickness of an atrial appendage A, and the long axis is set to be larger than the maximum width of the atrial appendage A. The size of the loop 4a before ligation is set so that an area sufficiently larger than the cross-sectional area of the atrial appendage A can be surrounded. The dimensions and area of the atrial appendage A are measured by image diagnosis (X-ray CT, MRI, ultrasound, etc.) before surgery.

The knot 4b of the ligature loop 4 is placed near an end of the flat ring-shaped loop 4a in the long axis direction. When the wire constituting the ligature loop 4 is pulled from the proximal end of the loop shaft 3 to conduct ligation, the wire constituting the ligature loop 4 at a distal end of the loop shaft 3 is withdrawn into the inner hole 2 of the loop shaft 3, and the knot 4b hits the distal end of the loop shaft 3 and stops.

From such a state, the wire constituting the ligature loop 4 is continued to be pulled so that the wire passing through the knot 4b moves relative to the knot 4b, and the loop 4a is contracted and tightened.

The operation of the atrial-appendage ligation surgical tool 1 according to this embodiment configured as described above is described below.

In order to ligate the base of the atrial appendage A by using the atrial-appendage ligation surgical tool according to this embodiment, first, a distal end opening of a sheath is placed inside a pericardium by causing the sheath to penetrate through the body surface tissue and the pericardial sac from the lower portion of the xiphoid process. In this state, the atrial-appendage ligation surgical tool 1, holding at its distal end the ligature loop 4 in a wide open state before ligation, is inserted into the sheath and advanced into the pericardium.

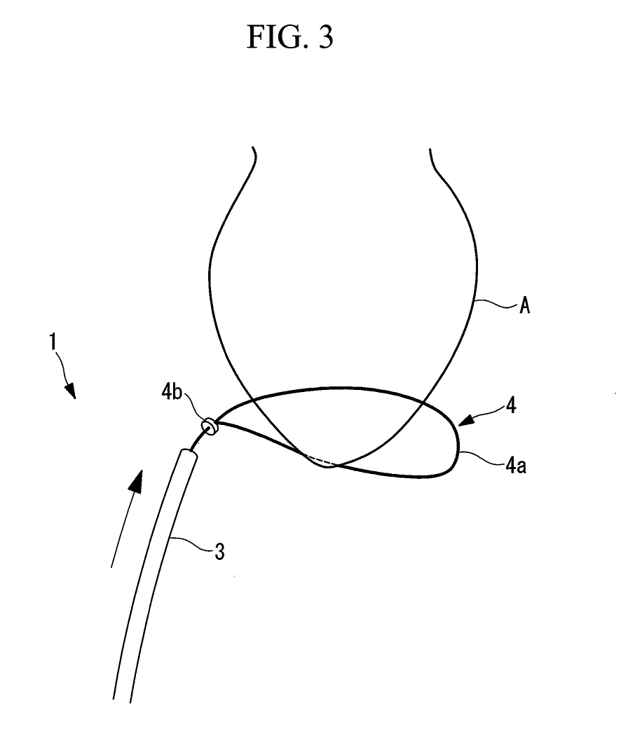

The loop shaft 3 of the atrial-appendage ligation surgical tool 1 advances smoothly into the pericardium while bending along the shape of the sheath. In such a state, the loop shaft 3 is manipulated from the proximal end of the sheath outside the body while conducting observation with an endoscope separately inserted into the pericardium, so that, as shown in FIG. 3, the distal end of the loop shaft 3 approaches the tip of the atrial appendage A. The loop 4a is put around the atrial appendage A by, if needed, pinching and supporting the loop at a position opposite to the knot 4b of the ligature loop 4 with retention forceps (not shown in the drawing) separately guided into the pericardium through the sheath.

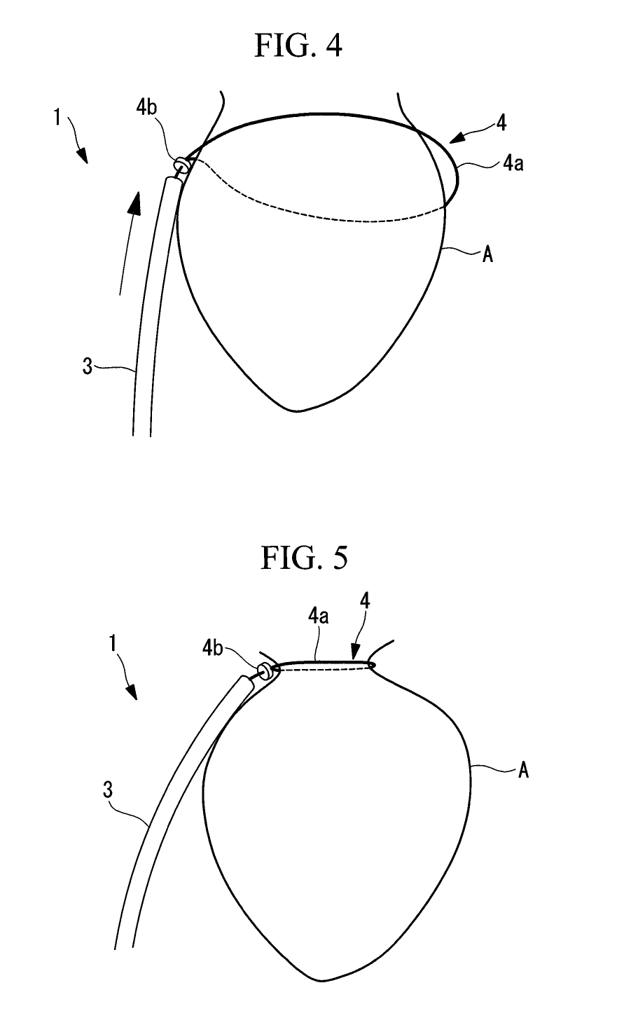

Since the loop 4a is given a tendency to form a flat ring shape, the loop 4a has a cross-sectional shape similar to that of the atrial appendage A, and thus the atrial appendage A can be inserted into the loop 4a without being deformed. Then, as shown in FIG. 4, when the loop shaft is pushed toward the distal end, the distal end of the loop shaft 3 moves along the edge of the atrial appendage A in the width direction, and the atrial appendage A is inserted into the loop 4a as the loop 4a is dragged by the loop shaft 3. As an assisting tool for inserting the atrial appendage into the loop 4a, retention forceps (not shown) inserted through a different sheath may be used to retain the loop 4a at the opposite side of the knot while the loop shaft 3 is being pushed.

That is, the loop 4a, which has been given a tendency to form a flat ring shape, allows the atrial appendage A to be continuously inserted thereinto without changing its topological relationship with respect to the atrial appendage A having a flat ring cross-sectional shape. Accordingly, the knot 4b placed near an end of the flat ring-shaped loop 4a in the long axis direction always remains at the end of the atrial appendage A in the width direction while the atrial appendage A is being inserted into the loop 4a. At the final stage, the knot 4b of the loop 4a is placed at an end of the base of the atrial appendage A in the width direction.

At this point, the atrial appendage A cannot be inserted into the loop 4a any further. Thus, the operator pulls the wire constituting the ligature loop 4 extending outward from the proximal end of the loop shaft 3 outside the body so that the loop 4a is contracted at the distal end of the loop shaft 3, as shown in FIG. 5.

As a result, the base of the atrial appendage A is ligated, and the thickness increases since the flat shape is narrowed in the width direction.

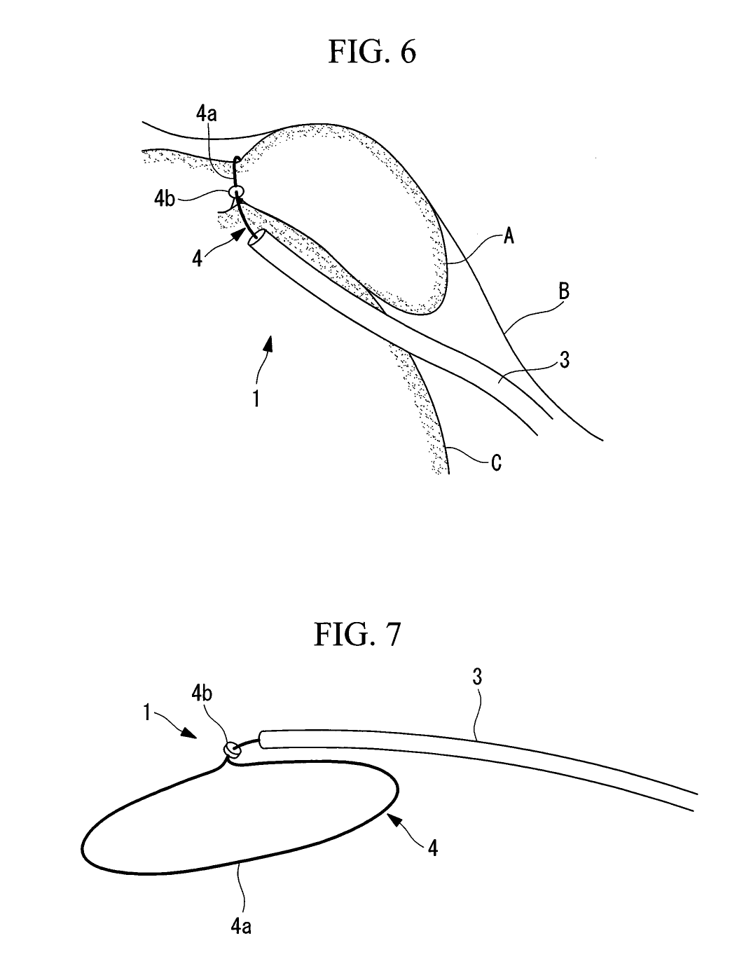

As described above, with the atrial-appendage ligation surgical tool 1 of this embodiment, the ease of inserting the atrial appendage A into the loop 4a when placing the loop 4a around the atrial appendage A can be improved because the loop 4a is given a tendency to form a flat ring shape. Moreover, after ligation, the knot 4b of the ligature loop 4 is located near an end of the atrial appendage A in the width direction; thus, as shown in FIG. 6, the atrial appendage A after ligation, having an increased thickness, creates a gap between a pericardial sac B and a heart C, and the knot 4b can be placed in this gap. Accordingly, there is an advantage that the knot 4b does not irritate the surrounding tissue.

In this embodiment, the case in which the ligature loop 4 is placed in such a manner that the distal end of the loop shaft 3 moves along the edge of the atrial appendage A in the width direction has been described. Alternatively, as shown in FIG. 7, a technique for guiding the distal end of the loop shaft 3 through the gap between the atrial appendage A and the pericardial sac B at the center position of the atrial appendage A in the width direction may be employed.

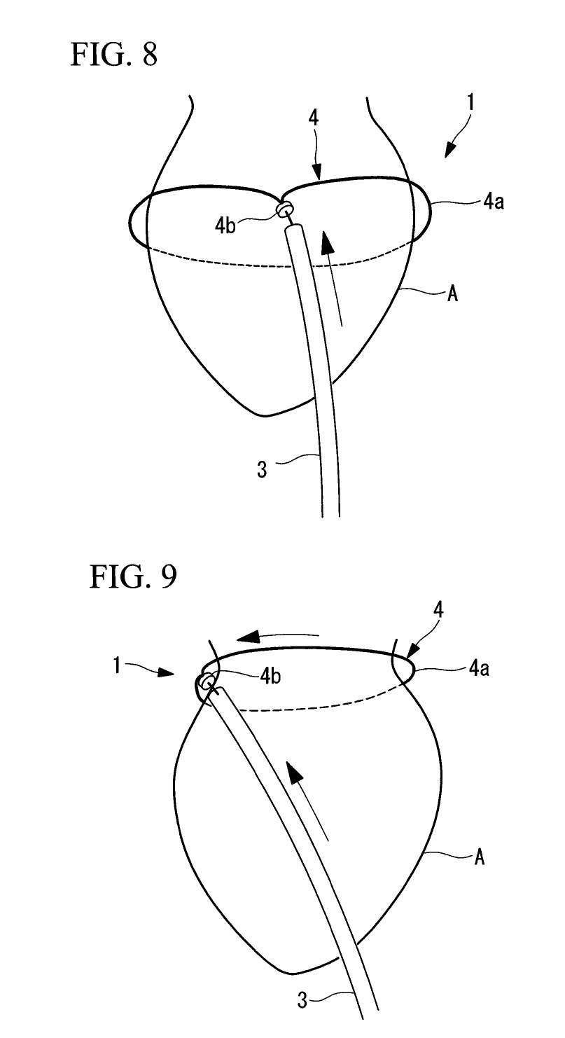

In such a case, as shown in FIG. 8, the knot 4b in the ligature loop 4 is preferably located near an end portion of the flat ring-shaped loop 4a in the short axis direction.

In other words, as shown in FIG. 8, in the case where the distal end of the loop shaft 3 moves toward the base of the atrial appendage A along the route that passes the center of the atrial appendage A in the width direction, the loop 4a, which is flat in the transverse direction, may be positioned under the knot 4b at the distal end of the loop shaft 3. In this manner, the shape of the loop 4a can be made to match the cross section of the atrial appendage A, and the ease of inserting the atrial appendage A into the loop 4a can be improved.

When the loop shaft 3 is pushed, the loop 4a is dragged and moved to the base of the atrial appendage A while maintaining the flat ring shape. When a pressing force is further applied to the loop shaft 3 that has reached the base of the atrial appendage A, as shown in FIG. 9, the distal end of the loop shaft 3 moves to an end of the atrial appendage A in the width direction while rotating the loop 4a in the circumferential direction of the atrial appendage A, and stops.

In such a state, the wire constituting the ligature loop 4 is pulled from the proximal end of the loop shaft 3 so as to contract the loop 4a, ligate the atrial appendage A, and place the knot 4b of the ligature loop 4 at an end of the atrial appendage A in the width direction, whereupon the ligation procedure ends. In other words, since the knot 4b is left near the end of the atrial appendage A in the width direction, the surrounding tissue is less likely to be irritated by the knot 4b.

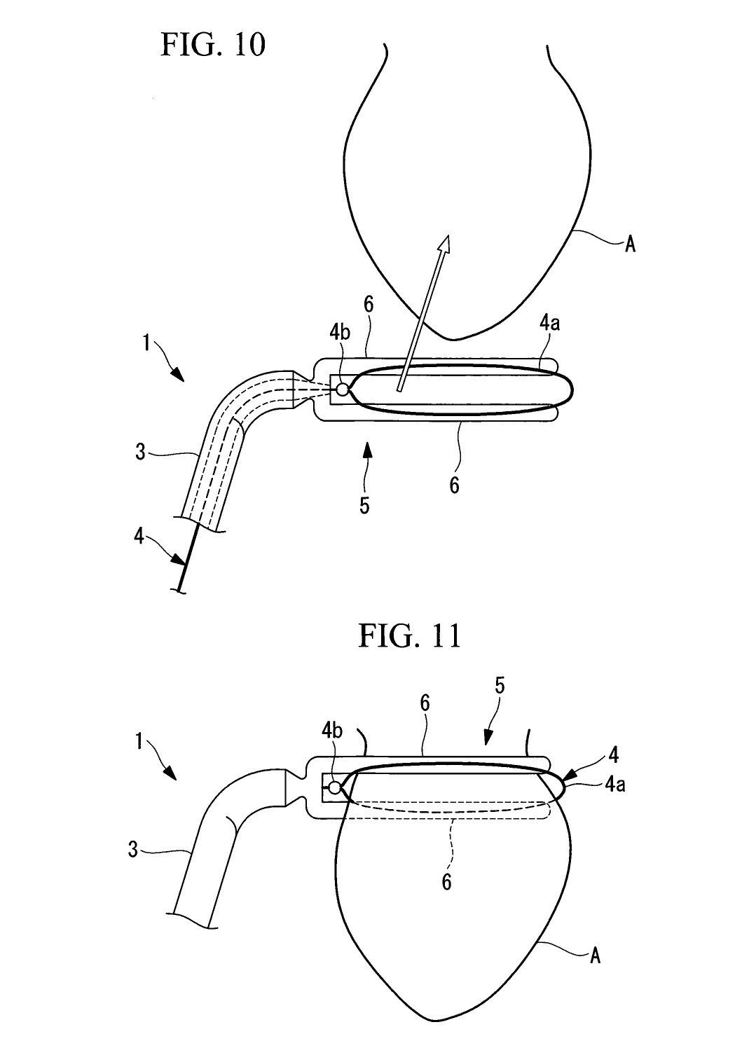

In this embodiment, an example of the atrial-appendage ligation surgical tool 1 that includes the loop shaft 3 and the ligature loop 4 has been described. Alternatively, as shown in FIG. 10, a pressing part 5 may be provided at the distal end of the loop shaft 3. The pressing part 5 has a bifurcated shape constituted by two straight rod-shaped contact portions 6 that branch from the loop shaft 3 side toward the distal end.

The loop shaft 3 is given a bending tendency. In a free state, as shown in FIG. 10, the pressing part 5 at the distal end is directed in a direction intersecting the longitudinal axis of the loop shaft.

The two contact portions 6 are arranged to be separated from each other by a gap that allows pinching of the atrial appendage A in the thickness direction.

The ligature loop 4 includes the loop 4a, which is given a tendency to form a flat ring shape, and the knot 4b is placed near an end of the loop 4a in the long axis direction.

In this case, the knot 4b is located near the branching point of the bifurcated shape constituted by the two contact portions 6.

With the atrial-appendage ligation surgical tool 1 having this structure, when the atrial appendage A is inserted into the loop 4a and the loop 4a is moved to the base of the atrial appendage A while the atrial appendage A is pinched by the two contact portions 6 in the thickness direction, the atrial appendage A, which has a similar flat cross section, can be smoothly inserted into the loop 4a which given a tendency to form a flat ring shape.

When the loop shaft 3 is moved in the insertion direction while having the pressing part 5 and the loop 4a placed at the atrial appendage A, and the two contact portions 6 pinching the atrial appendage A are pressed against the atrium, the vicinity of the base of the atrial appendage A becomes exposed. By placing the loop 4a around the exposed vicinity of the base of the atrial appendage A, it becomes possible to ligate the atrial appendage A at a position as close to the base as possible.

Since the two contact portions 6 cause the surface of the atrial appendage A to be indented in the thickness direction, the loop 4a does not easily come off toward the tip of the atrial appendage A when the loop 4a is contracted during ligation, and the atrial appendage A can be more reliably ligated at a position near the base.

At this point, as shown in FIG. 11, the branching point of the bifurcated shape of the pressing part 5, located at a position where the two contact portions 6 pinch the atrial appendage A in the thickness direction, is placed at an end of the atrial appendage A in the width direction. Thus, the knot 4b placed near the branching point is also located at the end of the atrial appendage A in the width direction. Accordingly, the knot 4b after ligation is also positioned at the end of the atrial appendage A in the width direction. Thus, ligation can be ended while having the knot 4b placed at a position where it rarely irritates the surrounding tissue.

The pressing part 5 may be of an immovable type or may be of an openable type with which the gap between the contact portions 6 can be changed. The ease of inserting the atrial appendage A can be improved by widening the gap between the contact portions 6, and the atrial appendage A can be pinched and the surface of the atrial appendage A can be indented by narrowing the gap between the contact portions 6.

### REFERENCE SIGNS LIST

A atrial appendage
1 atrial-appendage ligation surgical tool
2 inner hole
3 loop shaft
4 ligature loop
4a loop
4b knot
5 pressing part
6 contact portion

1. An atrial-appendage ligation surgical tool, comprising:
a tubular loop shaft having an inner hole that penetrates therethrough in a longitudinal direction; and
a ligature loop arranged to be in an inserted state in the inner hole of the loop shaft and formed of a wire that forms a loop closed with a knot at a distal end of the loop shaft, the loop being contractible in size by being pulled from a proximal end of the loop shaft,
wherein the ligature loop is given a tendency to form a flat ring-shaped loop having a size an atrial appendage is insertable when in a free state, and the knot is placed near an end of the loop in a long axis direction thereof.

2. An atrial-appendage ligation surgical tool, comprising:
a tubular loop shaft having an inner hole that penetrates therethrough in a longitudinal direction; and
a ligature loop arranged to be in an inserted state in the inner hole of the loop shaft and formed of a wire that forms a loop closed with a knot at a distal end of the loop shaft, the loop being contractible in size by being pulled from a proximal end of the loop shaft,
wherein the ligature loop is given a tendency to form a flat ring-shaped loop having a size an atrial appendage is insertable when in a free state, and the knot is placed near an end of the loop in a short axis direction thereof.

3. An atrial-appendage ligation surgical tool, comprising:
a tubular loop shaft having an inner hole that penetrates therethrough in a longitudinal direction;
a ligature loop arranged to be in an inserted state in the inner hole of the loop shaft and formed of a wire that forms a loop closed with a knot at a distal end of the loop shaft, the loop being contractible in size by being pulled from a proximal end of the loop shaft; and
a bifurcated pressing part disposed at the distal end of the loop shaft, and having two rod-shaped contact portions that can be placed at positions that allow pinching of an atrial appendage in a thickness direction, the contact portions branching from the loop shaft side toward the distal end,
wherein the knot in the ligature loop is placed near a branching point of the contact portions.
